(19) United States
(12) Patent Application Publication
(10) Pub. No.: US 2022/0233414 A1
(43) Pub. Date: Jul. 28, 2022

(54) MASCARA COMPOSITION AND METHOD OF USE

(71) Applicant: L'ORÉAL, Paris (FR)

(72) Inventors: Christopher PANG, Nanuet, NY (US); Sylvie PORET-FRISTOT, Clark, NJ (US)

(73) Assignee: L'ORÉAL, Paris (FR)

(21) Appl. No.: 17/159,574

(22) Filed: Jan. 27, 2021

(51) Int. Cl. A61K 8/19; A61K 8/92; A61K 8/81; A61K 8/06; A61Q 1/10

(52) U.S. Cl. CPC A61K 8/19; A61K 8/92; A61K 8/8117; A61K 8/8152; A61K 8/062; A61K 2800/88; A61K 2800/43; A61K 2800/47; A61K 2800/48; A61K 2800/524; A61K 2800/805; A61Q 1/10

(57) ABSTRACT

A method for improving the curl of eyelashes is provided. A composition is applied to eyelashes, and a magnetic element is then moved above the eyelash in the direction of desired curl. The disclosed composition, which may also be provided separately or in a kit along with a magnetic element for use with the disclosed method, requires three components: (i) one or more iron oxides in a total amount of at least 20% by weight, (ii) one or more latex polymers present in a total amount of at least 6% by weight; and (iii) one or more waxes having a melting point Tₘ≥80° C.

### TECHNICAL FIELD

The present invention relates to compositions and techniques for curling eyelashes, and specifically to mascara compositions that create curl which can be further enhanced through the use of magnets.

### BACKGROUND

Mascaras are traditionally used to improve the perceived aesthetics of a person's eyelashes. Such aesthetic qualities include the degree of curl. Conventional Mascara utilize waxes, surfactant, and film formers to improve the curl of the eyelashes. However, such techniques are inherently limited by available chemistries and the biological properties of a given person's eyelashes.

These mascaras must also provide high degrees of water and sebum resistance, yet be flexible enough that they will not significantly flake or crack as the eyelashes move or bend.

Therefore, a technique for enhancing the curling eyelashes, that avoids some of the inherent biological or chemical limitations, is desirable.

### BRIEF SUMMARY

A specific combination of materials provides a significant improvement in the curl of lashes when a magnet is used after application.

Specifically, a first aspect of the present disclosure is a method for improving the curl of eyelashes, where the composition includes (i) one or more iron oxides in a total amount of at least 20% by weight, (ii) one or more latex polymers present in a total amount of at least 6% by weight; and (iii) one or more waxes having a melting point Tₘ≥80° C. After application, within a first period of time (such as within 30 seconds), a magnetic element should be moved to a position above the eyelash in the direction of desired curl. In some embodiments, it may also be advantageous to repeatedly move the magnetic element in a direction substantially parallel (and/or substantially perpendicular) to the orientation of the eyelash while the magnetic element is above the eyelash in the direction of curl. The method according to claim 1, wherein the ratio of the one or more active latex polymers to the weight of waxes is between 0.76 and 1.0.

Optionally, the one or more active latex polymers comprise a styrene/acrylates/ammonium methacrylate copolymer. In various embodiments, the latex polymers are present in an amount between 8% and 16% by weight, at least 10% by weight, or at least 12% by weight.

In some embodiments, the one or more iron oxides are present in an amount between 20% and 50% by weight. In some embodiments, at least one iron oxide is uncoated.

In some embodiments, the one or more waxes with a melting temperature above 80° C. are present in a total amount of at least 1% by weight. Optionally, the aqueous cosmetic composition further comprises at least one wax having a melting point Tₘ<80° C.

Optionally, the aqueous cosmetic composition may also comprise: (i) a cellulosic polymer, a cellulose or rayon fiber, or a combination thereof; (ii) at least one additional colorant; (iii) at least one filler agent; (iv) at least one surfactant; and/or (v) at least one preservative.

Another aspect of the present disclosure is a mascara composition, which as described previously includes (i) one or more iron oxides in a total amount of at least 20% by weight, (ii) one or more latex polymers present in a total amount of at least 6% by weight; and (iii) one or more waxes having a melting point Tₘ≥80° C.

Another aspect of the present disclosure is a kit containing the mascara composition and a magnetic element that allows a user to magnetically interact with the aqueous cosmetic composition when the aqueous cosmetic composition is applied to an eyelash. Instructions for use may also be included in the kit.

### BRIEF DESCRIPTION OF THE DRAWINGS

FIG. 1 is a graph illustrating the impact of the concentration of latex polymer on maximum curl in a formula.

FIG. 2 is an illustration of an embodiment of a system according to the present invention, including an eyelash, eyelid, and magnet.

FIG. 3 is an illustration of an embodiment of a magnetic element adapted for use by a user.

### DETAILED DESCRIPTION

As used herein, articles such as “a” and “an” when used in a claim, are understood to mean one or more of what is claimed or described.

As used herein, the term “about [a number]” is intended to include values rounded to the appropriate significant digit. Thus, “about 1” would be intended to include values between 0.5 and 1.5, whereas “about 1.0” would be intended to include values between 0.95 and 1.05.

As used herein, the term “at least one” means one or more and thus includes individual components as well as mixtures/combinations.

As used herein, the term “free [of an ingredient]” means that the identified ingredient is only present in an amount below its detectable limit, and preferably that the composition contains 0% of the identified ingredient.

As used herein, the terms “include”, “includes” and “including” are meant to be non-limiting.

As used herein, the term “substantially free [of an ingredient]” means that the composition contains less than 1% of the identified ingredient.

Disclosed is a method, composition, and kit for providing semi-permanent curl to eyelashes. The method generally involves three steps: providing a particular aqueous cosmetic composition, applying the composition to an eyelash, and then exposing the eyelash to a magnetic element held above the eyelash in the direction of curl.

Step 1. Providing the Composition.

The method generally begins by providing the particular aqueous cosmetic composition that will be applied to an eyelash. This disclosed composition comprises at least three categories of materials: (1) one or more ferromagnetic pigments in a total amount of at least 20% by weight; (2) one or more latex polymers present in an amount such that the total solids content of all latex polymers is at least 6% by weight; and (3) one or more waxes having a melting point Tₘ≥80° C. (which are sometimes referred to herein as “high melting point” waxes). Each of these components will be discussed in turn below.

1. Ferromagnetic Pigments

As used herein, the term “ferromagnetic pigment” means a cosmetically acceptable material that exhibits ferromagnetic properties, including being attracted by a magnet. These materials include metals and metal oxides, such as iron oxide. A particularly suitable pigment is black iron oxide (CI 77499) which is a ferrous-ferric oxide. The pigment may be pearlescent.

The disclosed compositions utilize one or more iron oxides.

Particularly suitable pigments are pearlescents comprising iron oxides Fe₃O₄. Pigments with magnetic properties are available, for example, under the trade names Colorona Blackstar Blue, Colorona Blackstar Green, Colorona Blackstar Gold, Colorona Blackstar Red, Cloisonne Nu Antique Super Green, Microna Matte Black (17437), Mica Black (17260), Flamenco Twilight Red, Flamenco Twilight Green, Flamenco Twilight Gold, Flamenco Twilight Blue, Timica Nu Antique Silver 110 AB, Timica Nu Antique Gold 212 GB, available from Colorona Patina Silver (17289) and Colorona Patina Gold (117288), or Engelhard Timica Nu-Antique Copper 340 AB, Timica Nu Antique Bronze 240 AB, Cloisonne Nu Antique Green 828 CB, Cloisonne Nu Antique Blue 626 CB, Gemtone Moonstone G 004, Cloisonne Nu Antique Red 424 CB, Chroma-Lite Black (4498), Cloisonne Nu Antique Rouge Flambe (code 440 XB), Cloisonne Nu Antique Bronze (240 XB), Cloisonne Nu Antique Gold (222 CB), and Cloisonne Nu Antique Copper (340 XB).

As further examples that may be part of the formulation of the composition, mention may be made of particles of black iron oxide, for example the product name Sicovit noir E172 sold by BASF.

The iron oxides may be coated or uncoated. For example, in some embodiments, one or more of the iron oxides are uncoated.

In some embodiments, the ferromagnetic pigment may be chemically or physically coupled to a supporting material (e.g., a polymer, silica, mica, etc.) in order to provide the desired aesthetic effect (such as a matte, pearled, satin, or metallic effect).

Preferably, the ferromagnetic pigments comprise at least 50%, at least 60%, at least 70%, at least 80%, at least 90%, or 100% of all colorants in the composition.

The ferromagnetic pigments should be present in a total amount of at least 20% by weight of the composition. In some embodiments, the ferromagnetic pigments are present in a total amount≥20%, ≥25%, ≥30%, ≥35%, ≥40%, or ≥45% by weight, and ≤50%, ≤45%, ≤40%, ≤35%, ≤30%, or ≤25% by weight, including all ranges and subranges thereof. In some embodiments, the ferromagnetic pigments are preferably present in a total amount of between 20% and 50% by weight, between 30% and 50% by weight, or between 40% and 50% by weight.

2. Latex Polymers

The disclosed compositions utilize one or more latex polymers.

Such latex polymers include, e.g., acrylate latex polymers and/or polyurethane latex polymers. In some embodiments, latex polymers consist of a single acrylate latex polymer. In some embodiments, the one or more latex polymers consist of a plurality of acrylate latex polymers. In some embodiments, the one or more latex polymers consist of a single polyurethane latex polymer. In some embodiments, the one or more latex polymers consist of a plurality of polyurethane latex polymers. In some embodiments, the one or more latex polymers consist of a plurality of polyurethane latex polymers. In some embodiments, the one or more latex polymers consist of one acrylate latex polymer and one polyurethane latex polymer. In some embodiments, the one or more latex polymers consist of at least one acrylate latex polymer and at least one polyurethane latex polymer.

Acrylate latex polymers can include, but are not limited to, those resulting from the homopolymerization or copolymerization of monomers chosen from (meth)acrylics, (meth)acrylates, (meth)acrylamides and/or vinyl homopolymers or copolymers. The term “(meth)acryl” and variations thereof, as used herein, means acryl or methacryl.

The (meth)acrylic monomers may be chosen from, for example, acrylic acid, methacrylic acid, citraconic acid, itaconic acid, maleic acid, fumaric acid, crotonic acid, and maleic anhydride. Additional non-limiting examples of (meth)acrylic monomers include C1-C8 alkyl (meth)acrylic, such as, for example, methyl (meth)acrylic, ethyl (meth)acrylic, propyl (meth)acrylic, isopropyl (meth)acrylic, butyl (meth)acrylic, tert-butyl (meth)acrylic, pentyl(meth) acrylic, isopentyl (meth)acrylic, neopentyl (meth)acrylic, hexyl (meth)acrylic, isohexyl (meth)acrylic, 2-ethylhexyl (meth)acrylic, cyclohexyl (meth)acrylic, isohexyl (meth)acrylic, heptyl (meth)acrylic, isoheptyl (meth)acrylic, octyl (meth)acrylic, isooctyl (meth)acrylic, as well as combinations of any of the above.

The esters of (meth)acrylic monomers may be, by way of non-limiting example, C1-C8 alkyl (meth)acrylates such as methyl (meth)acrylate, ethyl (meth)acrylate, propyl (meth)acrylate, isopropyl (meth)acrylate, butyl (meth)acrylate, tert-butyl (meth)acrylate, pentyl(meth) acrylate, isopentyl (meth)acrylate, neopentyl (meth)acrylate, hexyl (meth)acrylate, isohexyl (meth)acrylate, 2-ethylhexyl (meth)acrylate, cyclohexyl (meth)acrylate, isohexyl (meth)acrylate, heptyl (meth)acrylate, isoheptyl (meth)acrylate, octyl (meth)acrylate, isooctyl (meth)acrylate, allyl (meth)acrylate, and combinations thereof. Additional and non-limiting examples include C1-C8 alkoxy (meth)acrylates, such as methoxy (meth)acrylate, ethoxy (meth)acrylate, propyl oxide (meth)acrylate, isopropyl oxide (meth)acrylate, butyl oxide (meth)acrylate, tert-butyl oxide (meth)acrylate, pentyl oxide (meth) acrylate, isopentyl oxide (meth)acrylate, neopentyl oxide (meth)acrylate. The esters may be, by way of non-limiting example, C2-C6 hydroxy alkyl (meth)acrylates, such as hydroxy ethyl (meth)acrylate, 2-hydroxypropyl (meth)acrylate, glycidyl (meth)acrylate, ethylene glycol di(meth)acrylate, polyethylene glycol mono(meth)acrylate, 1,4-butane diol di(meth)acrylate, 1,6,hexane diol di(meth)acrylate, and any combination thereof. The esters may be, by way of non-limiting example, aryl (meth)acrylates such as benzyl (meth)acrylate, phenyl (meth)acrylate, and any combination thereof. The esters can further contain amino groups such as aminoethyl (meth)acrylate, N,N-dimethylaminoethyl (meth)acrylate, N,N-dimethylaminopropyl (meth)acrylate, N,N-dimethylaminodimethylpropyl (meth)acrylate, N,N-diethyleaminoethyl (meth)acrylate, and N,N,N-trimethylaminoethyl (meth)acrylate; and salts of the ethylenic amines.

According to at least certain exemplary embodiments, the alkyl group of the esters may be either fluorinated or perfluorinated, e.g. some or all of the hydrogen atoms of the alkyl group are substituted with fluorine atoms. The monomers can also be fluorine-containing monomers, such as, by way of non-limiting example, trifluoroethyl methacrylate, 2,2,3,3-tetrafluoropropyl methacrylate, 2,2,3,3,4,4-hexafluorobutyl methacrylate, perfluorooctyl methacrylate and perfluorooctyl acrylate; and silicone macromonomers.

The amides of (meth)acrylic monomers can, for example, be made of (meth)acrylamides, and especially N-alkyl (meth)acrylamides, in particular N—(C1-C12) alkyl (meth)acrylates such as N-ethyl (meth)acrylamide, N-t-butyl (meth)acrylamide, N-t-octyl (meth)acrylamide, N-methylol (meth)acrylamide and N-diacetone (meth)acrylamide, and any combination thereof.

The vinyl monomers can include, but are not limited to, vinyl cyanide compounds such as acrylonitrile and methacrylonitrile; vinyl esters such as vinyl formate, vinyl acetate, vinyl propionate, vinyl neodecanoate, vinyl pivalate, vinyl benzoate and vinyl t-butyl benzoate, triallyl cyanurate; vinyl halides such as vinyl chloride and vinylidene chloride; aromatic mono- or divinyl compounds such as styrene, α-methylstyrene, chlorostyrene, alkylstyrene, divinylbenzene and diallyl phthalate, and combination thereof. Other non-limiting ionic monomers can include para-styrensulfonic, vinyl sulfonic, 2-(meth)acryloyloxy ethyl sulfonic, 2-(meth)acrylamido-2-methylpropylsulfonic acids.

The list of monomers given is not limiting, and it should be understood that it is possible to use any monomer known to those skilled in the art which includes acrylic and/or vinyl monomers (including monomers modified with a silicone chain).

Silicone acrylic polymers may also optionally be used as vinyl polymer in at least one exemplary and non-limiting embodiment.

In at least certain, non-limiting exemplary embodiments, acrylic latex polymers may be chosen from aqueous dispersions of Methacrylic Acid/Ethyl Acrylate copolymer (INCI: Acrylates Copolymer, such as Luviflex® Soft by BASF), PEG/PPG-23/6 Dimethicone Citraconate/C10-30 Alkyl PEG-25 Methacrylate/Acrylic Acid/Methacrylic Acid/Ethyl Acrylate/Trimethylolpropane PEG-15 Triacrylate copolymer (INCI: Polyacrylate-2 Crosspolymer, such as Fixate Superhold™ by Lubrizol), Styrene/Acrylic copolymer (such as Neocryl® A-1120, DSM), Ethylhexyl Acrylate/Methyl Methacrylate/Butyl Acrylate/Acrylic Acid/Methacrylic Acid copolymer (INCI: Acrylates/Ethylhexyl Acrylate Copolymer, such as Daitosol 5000SJ, Daito Kasei Kogyo), Acrylic/Acrylates Copolymer (INCI name: Acrylates Copolymer, such as Daitosol 5000AD, Daito Kasei Kogyo), and Acrylic copolymers and Acrylates Copolymers, such as those known under the tradenames VINYSOL 2140 (Daido Chemical), ACULYN™ 33 (Dow Chemical), LUVIMER® MAE (BASF), or BALANCE CR (AKZO NOBEL).

In a preferred embodiment, the acrylate latex polymer comprises a styrene/acrylates/ammonium methacrylate copolymer, such as those sold under the SYNTRAN® brand name.

Polyurethane latex polymers may include, but are not limited to, the reaction products of (i), (ii), and/or (iii), defined below.

Reaction product (i) may be any prepolymer according to the formula:

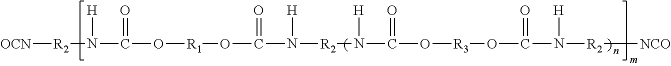

wherein R1 is chosen from bivalent radicals of a dihydroxyl functional compound, R2 is chosen from hydrocarbon radicals of an aliphatic or cycloaliphatic polyisocyanate, and R3 is chosen from radicals of a low molecular weight diol, optionally substituted with ionic groups, n ranges from about 0 to about 5, and m is greater than about 1.

Suitable dihydroxyl compounds for providing the bivalent radical R1 include those having at least two hydroxy groups, and having number average molecular weights ranging from about 700 to about 16,000, such as, for example, from about 750 to about 5000. Non-limiting examples of the high molecular weight compounds include polyester polyols, polyether polyols, polyhydroxy polycarbonates, polyhydroxy polyacetals, polyhydroxy polyacrylates, polyhydroxy polyester amides, polyhydroxy polyalkadienes and polyhydroxy polythioethers. In various embodiments, polyester polyols, polyether polyols, and polyhydroxy polycarbonates may be chosen. Mixtures of such compounds are also within the scope of the disclosure.

The polyester diol(s) may optionally be prepared from aliphatic, cycloaliphatic, or aromatic dicarboxylic or polycarboxylic acids, or anhydrides thereof; and dihydric alcohols such as diols chosen from aliphatic, alicyclic, or aromatic diols.

The aliphatic dicarboxylic or polycarboxylic acids may be chosen from, for example: succinic, fumaric, glutaric, 2,2-dimethylglutaric, adipic, itaconic, pimelic, suberic, azelaic, sebacic, maleic, malonic, 2,2-dimethylmalonic, nonanedicarboxylic, decanedicarboxylic, dodecanedioic, 1,3-cyclohexanedicarboxylic, 1,4-cyclohexanedicarboxylic, 2,5-norboranedicarboxylic, diglycolic, thiodipropionic, 2,5-naphthalenedicarboxylic, 2,6-naphthalenedicarboxylic, phthalic, terephthalic, isophthalic, oxanic, o-phthalic, tetrahydrophthalic, hexahydrophthalic or trimellitic acid.

The acid anhydrides may, in further exemplary embodiments, be chosen from o-phthalic, trimellitic or succinic acid anhydride or a mixture thereof. By way of non-limiting example only, the dicarboxylic acid may be adipic acid.

The dihydric alcohols may be chosen from, for example, ethanediol, ethylene glycol, diethylene glycol, triethylene glycol, trimethylene glycol, tetraethylene glycol, 1,2-propanediol, dipropylene glycol, tripropylene glycol, tetrapropylene glycol, 1,3-propanediol, 1,4-butanediol, 1,3-butanediol, 2,3-butanediol, 1,5-pentanediol, 1,6-hexanediol, 2,2-dimethyl-1,3-propanediol, 1,4-dihydroxycyclohexane, 1,4-dimethylolcyclohexane, cyclohexanedimethanol, 1,8-octanediol, 1,10-decanediol, 1,12-dodecanediol, neopentyl glycol, and mixtures thereof. The cycloaliphatic and/or aromatic dihydroxyl compounds may also be suitable as the dihydric alcohol(s) for the preparation of the polyester polyol(s).

The polyester diols may also be chosen from homopolymers or copolymers of lactones, which are, in at least certain embodiments, obtained by addition reactions of lactones or lactone mixtures, such as butyrolactone, ε-caprolactone and/or methyl-ε-caprolactone with the appropriate polyfunctional, e.g. difunctional, starter molecules such as, for example, the dihydric alcohols mentioned above. The corresponding polymers of ε-caprolactone may be chosen in at least some embodiments.

The polyester polyol, e.g. polyester diol, radical R1, may be obtained by polycondensation of dicarboxylic acids, such as adipic acid, with polyols, e.g. diols, such as hexanediol, neopentyl glycol, and mixtures thereof.

The polycarbonates containing hydroxyl groups comprise those known per se, such as the products obtained by reacting diols, such as (1,3)-propanediol, (1,4)-butanediol and/or (1,6)-hexanediol, diethylene glycol, triethylene glycol, or tetraethylene glycol with diaryl carbonates, for example diphenyl carbonate or phosgene.

Optional polyether polyols may be obtained in any known manner by reacting starting compounds which contain reactive hydrogen atoms with alkylene oxides, such as, for example, ethylene oxide; propylene oxide; butylene oxide; styrene oxide; tetrahydrofuran; or epichlorohydrin, or with mixtures of these alkylene oxides. In at least certain embodiments, the polyethers do not contain more than about 10% by weight of ethylene oxide units. For example, polyethers obtained without addition of ethylene oxide may be chosen.

Polyethers modified with vinyl polymers are also suitable according to various embodiments of the disclosure. Products of this type can be obtained by polymerization, for example, of styrene and acrylonitrile in the presence of polyethers, for example as described in U.S. Pat. Nos. 3,383,351; 3,304,273; 3,523,095; 3,110,695; and German patent 1 152 536 (each of which is incorporated herein in its entirety).

Among the polythioethers which may be chosen include the condensation products obtained from thiodiglycol per se and/or with other glycols, dicarboxylic acids, formaldehyde, aminocarboxylic acids, and/or amino alcohols. The products obtained are either mixed polythioethers, polythioether esters, or polythioether ester amides, depending on the co-components.

Optional polyacetals include but are not limited to the compounds which can be prepared from aldehydes, for example formaldehyde, and from glycols, such as diethylene glycol, triethylene glycol, ethoxylated 4,4′-(dihydroxy)diphenyl-dimethylmethane, and (1,6)-hexanediol. Polyacetals useful according to various non-limiting embodiments of the disclosure can also be prepared by polymerization of cyclic acetals.

Optional polyhydroxy polyesteramides and polyamines include, for example, the mainly linear condensation products obtained from saturated or unsaturated, polybasic carboxylic acids or anhydrides thereof, and from saturated or unsaturated, polyvalent amino alcohols, from diamines, or from polyamines, as well as mixtures thereof.

Optional monomers for the production of polyacrylates having hydroxyl functionality comprise acrylic acid, methacrylic acid, crotonic acid, maleic anhydride, 2-hydroxyethyl acrylate, 2-hydroxyethyl methacrylate, 2-hydroxypropyl acrylate, 2-hydroxypropyl methacrylate, 3-hydroxypropyl acrylate, 3-hydroxypropyl methacrylate, glycidyl acrylate, glycidyl methacrylate, 2-isocyanatoethyl acrylate, and 2-isocyanatoethyl methacrylate.

Mixtures of dihydroxy compounds can also be chosen.

Optional polyisocyanates for providing the hydrocarbon-based radical R2 include, for example, organic diisocyanates having a molecular weight ranging from about 100 to about 1500, such as about 112 to about 1000, or about 140 to about 400.

Optional diisocyanates are those chosen from the general formula R2(NCO)2, in which R2 represents a divalent aliphatic hydrocarbon group comprising from about 4 to 18 carbon atoms, a divalent cycloaliphatic hydrocarbon group comprising from about 5 to 15 carbon atoms, a divalent araliphatic hydrocarbon group comprising from about 7 to 15 carbon atoms, or a divalent aromatic hydrocarbon group comprising from about 6 to 15 carbon atoms. Examples of the organic diisocyanates which may be chosen include, but are not limited to, tetramethylene diisocyanate, 1,6-hexamethylene diisocyanate, dodecamethylene diisocyanate, cyclohexane-1,3-diisocyanate and cyclohexane-1,4-diisocyanate, 1-isocyanato-3-isocyanatomethyl-3,5,5-trimethylcyclohexane (isophorone diisocyanate or IPDI), bis(4-isocyanatocyclohexyl)-methane, 1,3-bis(isocyanatomethyl)cyclohexane and 1,4-bis(isocyanatomethyl)cyclohexane and bis(4-isocyanato-3-methylcyclohexyl)methane. Mixtures of diisocyanates can also be used.

In at least certain embodiments, diisocyanates are chosen from aliphatic and cycloaliphatic diisocyanates. For example, 1,6-hexamethylene diisocyanate, isophorone diisocyanate, and dicyclohexylmethane diisocyanate, as well as mixtures thereof may be chosen.

The use of diols, for example low molecular weight diols, R3, may in at least certain embodiments allow a stiffening of the polymer chain. The expression “low molecular weight diols” means diols having a molecular weight ranging from about 50 to about 800, such as about 60 to 700, or about 62 to 200. They may, in various embodiments, contain aliphatic, alicyclic, or aromatic groups. In certain exemplary embodiments, the compounds contain only aliphatic groups. The diols that may be chosen may optionally have up to about 20 carbon atoms, and may be chosen, for example, from ethylene glycol, diethylene glycol, propane-1,2-diol, propane-1,3-diol, butane-1,4-diol, 1,3-butylene glycol, neopentyl glycol, butylethylpropanediol, cyclohexanediol, 1,4-cyclohexanedimethanol, hexane-1,6-diol, bisphenol A (2,2-bis(4-hydroxyphenyl)propane), hydrogenated bisphenol A (2,2-bis(4-hydroxycyclohexyl)-propane), and mixtures thereof. For example, R3 may be derived from neopentyl glycol.

Optionally, the low molecular weight diols may contain ionic or potentially ionic groups. Suitable low molecular weight diols containing ionic or potentially ionic groups may be chosen from those disclosed in U.S. Pat. No. 3,412,054 (which is incorporated herein in its entirety). In various embodiments, compounds may be chosen from dimethylol-butanoic acid (DMBA), dimethylolpropionic acid (DMPA), and carboxyl-containing caprolactone polyester diol. If low molecular weight diols containing ionic or potentially ionic groups are chosen, they may, for example, be used in an amount such that less than about 0.30 meq of —COOH is present per gram of polyurethane in the polyurethane dispersion. In at least certain exemplary and non-limiting embodiments, the low molecular weight diols containing ionic or potentially ionic groups are not used.

Reaction product (ii) may be chosen from at least one chain extender according to the formula: H2N—R4-NH2

wherein R4 is chosen from alkylene or alkylene oxide radicals, said radicals not being substituted with ionic or potentially ionic groups.

Reaction product (ii) may optionally be chosen from alkylene diamines, such as hydrazine, ethylenediamine, propylenediamine, 1,4-butylenediamine and piperazine; and alkylene oxide diamines such as dipropylamine diethylene glycol (DPA-DEG available from Tomah Products, Milton, Wis.), 2-methyl-1,5-pentanediamine (Dytec A from DuPont), hexanediamine, isophoronediamine, and 4,4-methylenedi(cyclohexylamine), and the DPA-series of ether amines available from Tomah Products, Milton, Wis., including dipropylamine propylene glycol, dipropylamine dipropylene glycol, dipropylamine tripropylene glycol, dipropylamine poly(propylene glycol), dipropylamine ethylene glycol, dipropylamine poly(ethylene glycol), dipropylamine 1,3-propanediol, dipropylamine 2-methyl-1,3-propanediol, dipropylamine 1,4-butanediol, dipropylamine 1,3-butanediol, dipropylamine 1,6-hexanediol and dipropylamine cyclohexane-1,4-dimethanol, and mixtures thereof.

Reaction product (iii) may be chosen from at least one chain extender according to the formula: H2N—R5-NH2

wherein R5 is chosen from alkylene radicals substituted with ionic or potentially ionic groups. In at least certain exemplary embodiments, the compounds may have an ionic or potentially ionic group and two isocyanate-reactive groups.

As used herein, ionic or potentially ionic groups may include groups comprising ternary or quaternary ammonium groups, groups convertible into such groups, carboxyl groups, carboxylate groups, sulphonic acid groups, and sulphonate groups. At least partial conversion of the groups convertible into salt groups of the type mentioned may take place before or during the mixing with water. Specific compounds include diaminosulphonates, such as for example the sodium salt of N-(2-aminoethyl)-2-aminoethanesulphonic acid (AAS) or the sodium salt of N-(2-aminoethyl)-2-aminopropionic acid.

In at least certain embodiments, R5 represents an alkylene radical substituted with sulphonic acid or sulphonate groups. By way of example only, the compound is chosen from sodium salts of N-(2-aminoethyl)-2-aminoethanesulphonic acid.

By way of non-limiting example, such polyurethane latex polymers include, but are not limited to, reaction products of a prepolymer comprising a dihydroxyl compound, a polyisocyanate, and a low molecular weight diol and at least two diamine compounds and wherein the composition is substantially free of triethanolamine stearate such as, for example, those sold under the BAYCUSAN® name by Bayer such as, for example, BAYCUSAN® C1000 (INCI name: Polyurethane-34), BAYCUSAN® C1001 (INCI name: Polyurethane-34), BAYCUSAN® C1003 (INCI name: Polyurethane-32), BAYCUSAN® C1004 (INCI name: Polyurethane-35) and BAYCUSAN® C1008 (INCI name: Polyurethane-48). In various exemplary embodiments, polyurethane latexes may be chosen from, but are not limited to, aqueous polyurethane dispersion of Isophthalic Acid/Adipic Acid/Hexylene Glycol/Neopentyl glycol/Dimethylolpropanoic Acid/Isophorone Diisocyanate copolymer (INCI name: Polyurethane-1, such as Luviset® P.U.R, BASF), aliphatic polyurethane and aliphatic polyester polyurethane (such as the Neorez® series, DSM, such as Neorez® R989, INCI name: Polycarbamyl Polyglycon Ester).

It is understood that a polyurethane latex polymer is usually provided as aqueous polyurethane dispersions comprising the polyurethane latex polymer.

The latex polymers may include a polyacrylic latex, a polyacrylate latex, a polystyrene latex, a polyester latex, a polyamide latex, a polyurea latex, a polyurethane latex, an epoxy resin latex, a cellulose-acrylate latex, and their copolymers.

In various embodiments according to the disclosure, it may be possible to choose a polymer that comprises both acrylate and polyurethane parts at the molecular level.

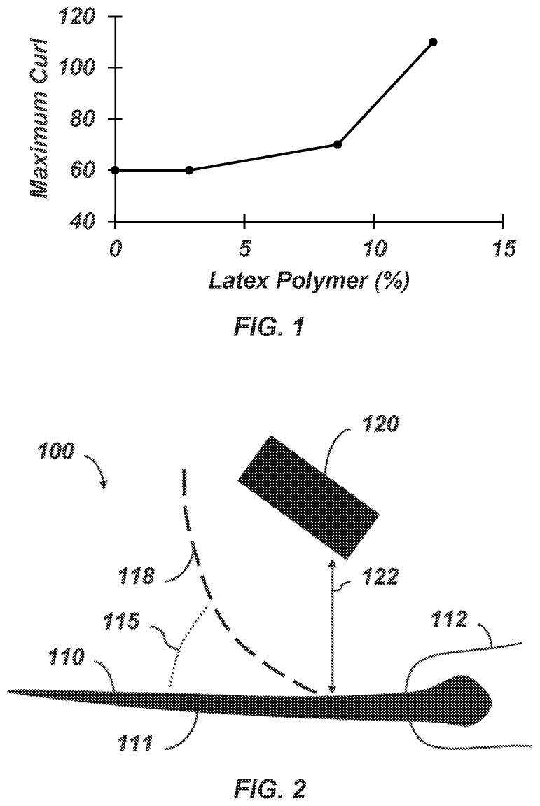

The amount of latex polymer present in the application is one critical component to the disclosed aqueous compositions. FIG. 1 is a graph that shows the maximum curl experienced by an eyelash when a formula containing 50% iron oxides, 1-3% waxes with a melting point≥80° C., and a varying amount of latex polymer is applied. To measure the maximum curl, each mascara composition was tested using artificial eyelashes, held in place with a thin flat clamp. The mascaras were applied with an applicator, and some of those test eyelashes were then exposed to a magnetic field from a hand-held magnet which was moved over the eyelashes 20 times, for a total exposure time of about 1 minute. The maximum curl was measured by holding each clamp horizontally, and measuring the angles formed by the eyelashes with a protractor.

Surprisingly, to enhance the curl, the latex polymer must be present in a total amount of at least 6% by weight. Any less, and no enhancement occurs. Further, it is additionally unexpected that above 6% concentrations, the maximum curl measured at 50% iron oxide use levels increases as the amount of latex polymer increases.

As seen in FIG. 1, the maximum curl experienced by an eyelash begins to see an increase at 6% by weight latex polymer. It was found that, preferably, the amount of latex polymer used in a composition is between 8% and 16% by weight, and more preferably at least 10% by weight, and most preferably at least 12% by weight of the composition. In some embodiments, the amount of late polymer is between 10% and 16%. In some embodiments, the amount of latex polymer is between 12% and 16%.

3. High Melting Point Waxes

The disclosed compositions include one or more waxes having a melting point Tₘ≥80° C.

The high melting point waxes may include at least one synthetic polyethylene wax, or wax of natural origin.

Non-limiting examples of suitable polyethylene waxes with a high melting point (>80° C.) include an ethylene homopolymer or a copolymer of ethylene and of another copolymerizable monomer corresponding to the following formula (I):

CH2=CHR (I)

in which R represents a linear or branched alkyl chain which can be interrupted by mono- or polyoxyalkylene units, an aryl or aralkyl radical or —CH2COOH or —CH2CH2OH radical.

The alkyl radicals more particularly denote the methyl, ethyl, propyl, isopropyl, decyl, dodecyl and octadecyl radicals.

The mono- or polyoxyalkylene units preferably denote mono- or polyoxyethylene groups or mono- or polyoxypropylene groups.

The aryl radical is preferably a phenyl or tolyl radical.

The aralkyl radical is, for example, a benzyl or phenethyl radical.

The weight-average molar mass of the polyethylene wax with a high melting point according to the invention is preferably between approximately 400 and 1000, more particularly between approximately 400 and 700 and is preferably about 500.

According to a preferred embodiment of the compositions according to the invention, the wax as defined above is chosen from ethylene homopolymers, copolymers of ethylene and of propylene, copolymers of ethylene and of maleic anhydride or acid, or oxidized or ethoxylated polyethylenes.

Mention may in particular be made, among the ethylene homopolymers which can be used according to the invention, of those sold under the names of Polywax 500, Polywax 655 and Polywax 1000 by the company Petrolite.

Mention may be made, among the ethylene copolymers which can be used according to the invention, of the copolymers of ethylene and of propylene sold under the names Petrolite® by the company Petrolite, the copolymers of ethylene and of maleic anhydride sold under the names Ceramer® by the company Petrolite, the oxidized polyethylenes sold under the names Unilin® and Unicid® by the company Petrolite, and the ethoxylated polyethylenes sold under the names Unithox® by the company Petrolite.

In some embodiments, the polyethylene wax is an ethylene homopolymer wax.

Non-limiting examples of natural waxes include mineral, fossil, animal or vegetable waxes, or hydrogenated oils, fatty esters, fatty alcohols or polyoxyethylenated fatty alcohols which are solid at 25° C., including, e.g., microcrystalline waxes, ceresin, ozokerite, candelilla wax, carnauba wax, and/or hydrogenated castor oil.

According to a preferred embodiment of the invention, the high melting point wax comprises or consists of carnauba wax.

In some embodiments, a single high melting point wax is used. In some embodiments, a plurality of high melting point waxes are used.

The one or more waxes with a melting temperature above 80° C. are present in a total amount of at least 1% by weight. In preferred embodiments, the one or more waxes with a melting temperature above 80° C. are present in a total amount of between 1% and 16%. In some embodiments, the one or more waxes with a melting temperature above 80° C. are present in a total amount of ≥1%, ≥1.5%, ≥2%, ≥2.5%, or ≥3%, and ≤16%, ≤12%, or ≤8%, or any combination of ranges thereof.

4. Other Materials

The disclosed composition may contain one or more additional components, including, but not limited to: low melting point waxes, cellulosic polymers, cellulose or rayon fibers, other thickeners or polymers, additional colorants, filler agents, surfactants, preservatives, other additives, and combinations thereof.

Low Melting Point Waxes

In addition to high melting point waxes, other waxes may be present in the composition, including those having a melting point Tₘ<80° C. (sometimes referred to herein as “low melting point waxes” to distinguish them from the high melting point waxes described previously). In some embodiments, at least one low melting point wax has a melting point between 55° C. and 70° C. In some embodiments, at least one low melting point wax has a melting point between 40° C. and 55° C.

In some embodiments, the compositions are substantially free of low melting point waxes. In some embodiments, the composition contains one low melting point wax. In some embodiments, the composition contains a plurality of low melting point waxes. In some embodiments, the composition contains only two low melting point waxes. In some embodiments, the composition contains only three low melting point waxes.

Examples of low melting point waxes that are suitable for the invention, include hydrocarbon-based waxes, such as natural and/or synthetic beeswax, paraffin wax, isoparaffin wax, lanolin wax, esparto grass wax, berry wax, shellac wax, and sumac wax, orange wax, lemon wax, and mixtures thereof.

Fatty alcohols may be utilized as low melting point waxes, especially those containing linear or branched C₈-C₃₂ fatty chains, preferably C₋₁₆ to C₋₁₈ chains, such as cetyl alcohol, steryl alcohol, or cetearyl alcohol.

Fatty acids obtained by catalytic hydrogenation of animal or plant oils containing linear or branched C₈-C₃₂ fatty chains, preferably C₋₁₆ to C₋₁₈ chains, may also be used as the low melting point wax.

Other low melting point waxes include silicone waxes, such as C₃₀₋₄₅ alkyl dimethicone, C₃₀₋₄₅ alkyldimethylsilyl polypropylsilsesquioxane, or fluoro waxes.

Various natural or synthetic butters may be utilized as low melting point waxes, including cocoa butter, shea butter, or jojoba butter.

Low melting point waxes are typically present in the composition, though not required.

In various embodiments of the invention, the low melting point wax is present in an amount from about 0.5% to about 15% by weight, such as from about 3% to about 14% by weight, typically from about 5% to about 13% by weight.

Of particular concern is the ratio of the weight of the one or more active latex polymers in a composition to the weight of all waxes (low melting point waxes+high melting point waxes) in the composition is between 0.76 and 1.0.

Cellulosic Polymers/Natural or Synthetic Fibers

The aqueous cosmetic composition may also contain at least one cellulosic polymer, a natural or synthetic fiber, or a combination thereof. Representative examples of cellulosic polymers include, but are not limited to, hydroxyethylcellulose, hydroxypropylcellulose, methylcellulose, ethylhydroxyethylcellulose, carboxymethylcellulose, and quaternized cellulose derivatives, nitrocellulose, cellulose acetate, cellulose acetobutyrate or cellulose acetopropionate. Representative examples of natural fibers include, but are not limited to, cotton, silk, wool, and other keratin fibers. Representative examples of synthetic fibers include, but are not limited to, polyester, rayon, nylon and other polyamide fibers. Such fibers may be present in the compositions in an amount generally ranging from about 0.01% to about 10% by weight of the composition.

Other Thickeners or Polymers

The aqueous cosmetic composition may also contain at least one other thickener or polymer. Representative thickeners or other polymers include xanthan gum, guar gum, acacia senegal gum, hydroxypropyl guar, guar hydroxypropyl trimonium chloride, hydroxypropyl starch phosphate, ammonium acryloyldimethyltaurate/VP copolymer, optionally crosslinked and/or neutralized 2-acrylamido-2-methylpropanesulfonic acid polymers (AMPS) and copolymers, crosslinked anionic copolymers of acrylamide, crosslinked anionic copolymers of AMPS, emulsions of crosslinked anionic copolymers of acrylamide/nonionic surfactants, emulsions of AMPS/nonionic surfactants, or mixtures thereof. Other thickeners or other polymers include, e.g., liposoluble film-forming polymers such as polyalkylenes, including copolymers of C₂-C₂₀ alkenes, polybutene, alkylcelluloses with a linear or branched, saturated or unsaturated C₁-C₈ alkyl radical, for instance ethylcellulose and propylcellulose, copolymers of vinylpyrrolidone (VP) such as copolymers of vinylpyrrolidone and of C₂-C₄₀ alkene such as C₃-C₂₀ alkene. Among the VP copolymers which may be used herein, mention may be made, for example, of the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

Mention is also made of a mixture of copolymers of a C36 diacid condensed on diamine ethylene will be used; the terminal ester groups result from the esterification of the terminations of remaining acid by the cetylic, stearylic alcohol or mixtures thereof (also called cetylstearylic) (INCI name: Ethylenediamine/Stearyl Dimer Dilinoleate Copolymer).

These other thickeners or polymers, if present, are preferably used in an amount generally ranging from about 1% to about 15% by weight of the composition.

Additional Colorants

The aqueous cosmetic composition may also contain at least one additional colorant. These additional colorants are not intended to be ferromagnetic. In some embodiments, those additional colorants are pigments and/or dyes.

Colorants are preferably chosen from pulverulent materials, liposoluble dyes and water-soluble dyes, and mixtures thereof.

Preferably, the compositions according to the invention comprise at least one pulverulent colorant. The pulverulent colorants may be chosen from pigments and nacres, and preferably from pigments.

The pigments may be white or colored, inorganic and/or organic, and coated or uncoated. Among the inorganic pigments, mention may be made of metal oxides, in particular titanium dioxide, optionally surface-treated, zirconium, zinc or cerium oxide, and also titanium or chromium oxide, manganese violet, ultramarine blue, chromium hydrate and ferric blue. Among the organic pigments that may be mentioned are carbon black, pigments of D&C type and lakes based on cochineal carmine or on barium, strontium, calcium or aluminum.

The nacres may be chosen from white nacreous pigments such as mica coated with titanium or with bismuth oxychloride, colored nacreous pigments such as titanium mica with iron oxides, titanium mica with in particular ferric blue or chromium oxide, titanium mica with an organic pigment of the abovementioned type, and also nacreous pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto.

In some embodiments, the mascara comprises at least one non-ferromagnetic pigment.

The proportion of additional colorants in the composition, if present, is generally between 0.1 and 25% by weight, relative to the total weight of the composition, according to the coloration and intensity of the sought after coloration.

Filler Agents

The aqueous cosmetic composition may also contain at least one filler agent.

The filler agents may include those of natural or synthetic origin. Fillers include, but are not limited to, mineral powders, such as, talc, kaolin, mica, silica, silicates, alumina, zeolites, hydroxyapatite, sericite, titanium micas, barium sulphate, bismuth oxychloride, boron nitride; metal powders, such as, aluminum powder, vegetable powders, such as starch, maize, wheat or rice powders; and organic powders, such as, nylon, polyamide, polyester, polytetrafluoroethylene or polyethylene powders.

The various powders may be coated, for example with metal salts of fatty acids, amino acids, lecithin, collagen, silicone compounds, fluorinated compounds, or with any other Customary coating.

Fillers also include talc, mica, silica, kaolin, nylon powder, poly-beta-alanine powder and polyethylene powder, Teflon, lauroyllysine, starch, boron nitride, tetrafluoroethylene polymer powders, hollow microspheres such as Expancel (Nobel is Industrie), polytrap (Dow Corning) and silicone resin microbeads (Tospearls from Toshiba for example), precipitated calcium carbonate, magnesium carbonate and hydrocarbonate, hydroxyapatite, hollow silica microspheres (Silica Beads from Maprecos), glass or ceramic microcapsules. Mention may also be made of metal soaps derived from organic carboxylic acids having from 8 to 22 carbon atoms, preferably from 12 to 18 carbon atoms, for example zinc, magnesium or lithium stearate, zinc laurate and magnesium myristate.

The fillers may be chosen from those that are well-known to those skilled in the art, and that are commonly used in cosmetic compositions. Fillers include zinc oxide and titanium oxide, which are generally used in the form of particles, not exceeding a few microns in size; calcium carbonate; magnesium carbonate or magnesium hydrocarbonate; microcrystalline cellulose; silica; synthetic polymer powders such as polyethylene, polyesters (polyethylene isophthalate or terephthalate); and polyamides.

In some embodiments, the additional filler agents are present in an amount between 1% and 40% by weight.

Surfactants

The aqueous cosmetic composition may also contain at least one surfactant.

If a surfactant is used, the surfactant is at least one selected from zwitterionic, anionic, cationic and nonionic surfactants.

The surfactant is generally from 0.3% to 20% by weight, in particular from 0.5% to 15% by weight, more particularly from 1% to 10% by weight, based on the total weight of the composition.

Solvents

The aqueous cosmetic composition will generally water and may optionally contain other solvents.

Water is generally present in an amount between 20% and 60% by weight, such as between 30% and 60% by weight, such as between 40% and 55% by weight.

Other solvents may include, e.g., polyols or C1-C4 monoalcohols, including glycerol and ethanol.

Preservatives

The aqueous cosmetic composition may also contain at least one preservative. Representative examples of preservatives include alkyl para-hydroxybenzoates, wherein the alkyl radical has from 1, 2, 3, 4, 5 or 6 carbon atoms and preferably from 1 to 4 carbon atoms e.g., methyl para-hydroxybenzoate(methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben), and phenoxyethanol. Mixtures of preservatives are also useful, e.g., the mixture of methylparaben, ethylparaben, propylparaben and butylparaben sold under the name Nipastat by Nipa, the mixture of phenoxyethanol, methylparaben, ethylparaben, propylparaben and butylparaben, also sold by Nipa under the name Phenonip, and the mixture of phenoxyethanol, methylparaben, isopropylparaben, isobutylparaben and butylparaben, sold by ISP under the tradename Liquapar Optima. The preservative may be present in an amount generally ranging from about 0.01% to about 15% by weight of the composition.

Other Additives

The aqueous cosmetic composition may also contain one or more other cosmetically acceptable additives conventionally employed in the formulation of specific formulations. Such additional additives can in particular be chosen from antioxidants, vitamins, chelating agents, neutralizing agents, skin conditioning agents, and fragrances.

Step 2. Applying the Composition.

The aqueous cosmetic composition provided in step 1 is then applied to an eyelash. This may be done using an applicator (not shown) or any other appropriate technique for introducing the mascara to the eyelash. The eyelash may be a natural or synthetic eyelash.

Referring briefly to FIG. 2, a natural eyelash 110 is shown extending from an eyelid 112. When the composition is applied to the eyelash 110, the composition is typically applied to a single side of the eyelash, such as the underside 111 of the eyelash (the side of the eyelash that is closest to the eye), although it can be applied to other portions of the eyelash as well. In reference to FIG. 1, the composition is preferably applied to the surface of eyelash 110 that faces away from the direction the user wishes to curl the eyelashes. The application process typically bends the eyelash at least somewhat in the direction curl is desired (e.g., curling the lash away from the eye).

Step 3. Using a Magnetic Element.

Referring back to FIG. 2, once the composition has been applied, the eyelash will begin to curl. As seen in FIG. 2, the eyelash will begin to curl from its normal, uncurled position 110, curling upwards towards its final curled position 118, curling at a deviation angle 115 that can be observed and measured visually.

After application, a magnetic element 120 can be temporarily moved above the eyelash, or moved around above the eyelash, in the direction curl is desired.

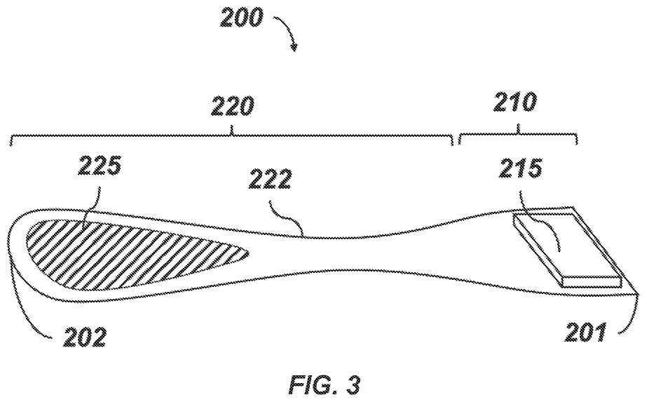

The magnetic element must be adapted to being moved above the eyelashes and utilized by the user. As such, while the magnetic element may simply be a magnet, a preferred embodiment utilizes a tool that comprises a magnet. Referring briefly to FIG. 3, tool 200 includes a functional portion 210 and a handle portion 220. The functional portion 210 contains a magnetic element 215 positioned adjacent to, or a short distance (e.g., ≤10 mm) from a first end 201 of the tool 200. The handle portion 220 may be shaped in an ergonomic manner (e.g., with a pinched waist 222) and may contain a textured portion 225 (e.g., to provide a grip) adjacent to, or a short distance (e.g., ≤10 mm) from a second end 202 of the tool 200.

The magnetic element attracts the ferromagnetic pigment, synergistically enhancing the curling efforts being undertaken by the latex polymer(s) and the high temperature wax(es).

Referring back to FIG. 1, the magnet is preferably kept at a distance 122 from the eyelash, sufficient to ensure the magnetic field (not shown in FIG. 1) from the magnet 120 can interact with the ferromagnetic particles that have been applied to the eyelash. The actual distance will therefore be determined, in part, by the strength of the magnet utilized. Preferably, the minimum distance 122 of the magnet to the eyelash is >0 mm, ≥about 2.5 mm, ≥about 5 mm, ≥about 7.5 mm, ≥about 10 mm, ≥about 12.5 mm, or ≥about 15 mm, and preferably the minimum distance is ≤about 50 mm, ≤about 45 mm, ≤about 40 mm, ≤about 35 mm, ≤about 30 mm, ≤about 25 mm, ≤about 20 mm, ≤15 mm, or ≤about 12.5 mm, or any combination thereof.

In some embodiments, the eyelashes are exposed to the magnetic field from the magnet within 30 seconds, and preferably within 10 seconds, after the mascara has been applied to the eyelash. In some embodiments, the eyelashes are exposed to the magnetic field from the magnet for a total time of between 5 seconds and 1 minute. In preferred embodiments, the eyelash is exposed to the magnetic field for at least 5 seconds, at least 10 seconds, at least 15 seconds, at least 20 seconds, at least 25 seconds, or at least 30 seconds, and no more than 60 seconds, no more than 50 seconds, no more than 40 seconds, or no more than 30 seconds.

While any strength magnet is envisioned, some embodiments utilize magnets with a gauss rating of between 100 and 20,000.

### Example

Four base composition was created (with increasing amounts of latex polymer used). See Table 1, below. An aqueous phase is produced by combining the aqueous components, except for the latex, and mixed at heated to high temperature between 75° C. and 80° C. An oil phase is produced by combining the oil-based components, then heating and mixing until all components have melted. The oil phase combined with the aqueous phase at high temperature and emulsified for at least 20 min and then cooled to room temperature. At room temperature, the latex was added and mixed until uniform. Afterwards, the final product was poured into an appropriate container.

TABLE 1
Base Compositions

| | Base 1 | Base 2 | Base 3 | Base 4 |
|---|---|---|---|---|
| Material | (% w/w) | (% w/w) | (% w/w) | (% w/w) |
| Latex Polymer | — | 1-3 | 6-9 | 10-16 |
| High Melting Point | 5-15 | 5-15 | 1-3 | 1-5 |
| Waxes | | | | |
| Low Melting Point | 5-15 | 5-15 | 5-10 | 5-15 |
| Waxes | | | | |
| Other Polymers | 5-15 | 5-15 | 3-10 | 3-10 |
| Surfactants | — | 5-15 | 3-10 | 5-15 |
| Preservative | — | 0-3 | 0-3 | 0-3 |
| Other Additives | 0-2 | 0-2 | 1-3 | 1-3 |
| Other Colorants | 0-1 | — | — | — |
| Filler Agents | 5-10 | — | 3-10 | — |
| Water | — | 40-60 | 30-50 | 20-40 |
| Other Solvents | 40-60 | — | 3-10 | 3-10 |

Each base composition was separated into 5 portions to which varying amounts of iron oxides was added to bring the total amount of iron oxides in each sample to 10%, 20%, 30%, 40%, and 50% by weight.

Evaluations. The results of the evaluations are summarized in Table 2, where a dash indicates the measurement was not taken.

Curl. Each composition was tested using artificial eyelashes, which were held in place with a thin flat clamp. The compositions were applied with an applicator, and some of those test eyelashes were then exposed to a magnetic field from a hand-held magnet which was moved over the eyelashes 20 times, for a total exposure time of about 1 minute. Each eyelash (including those that were not exposed to a magnet “Curl (No Magnet)” and those that were exposed “Curl (With Magnet)”) was evaluated at their initial lift angles. The evaluations were done by holding each clamp horizontally, and measuring the angles formed by the eyelashes with a protractor.

Flake Resistance. Each sample composition was applied to untreated human hair samples and completely dried. Each sample was then tied to see if the films formed on the hair samples crack or flaking when the hair is bent. The degree of cracking or flaking was then rated on a scale of 1-5, with 1 being none or minimal flaking (e.g., most desirable), and 5 being significant or complete flaking (e.g., least desirable).

Sebum and Water Resistance. Each sample composition was applied on fake eyelashes and then each fake eyelash was submerged into either artificial sebum or water as appropriate for 24 hours. The fake eyelashes were then brushed on water color papers 10 times to see if the mascara pigment transfers from the fake eyelashes to the water color paper. The degree to which the pigment transferred was then rated on a scale of 1-5, with 1 being none or minimal transfer (e.g., most desirable), and 5 being significant or total transfer (e.g., least desirable).

TABLE 2
Summary of Evaluations

| | % Iron | Curl | Curl | | | |
|---|---|---|---|---|---|---|
| Base | Oxide | (No Magnet) | (With Magnet) | Flake | Sebum | Water |
| 1 | 10% | 0 | 0 | 1 | 2 | 1 |
| | 20% | 0 | 20-35 | 2 | 5 | 1 |
| | 30% | 10 | 45-55 | 3 | 5 | 1 |
| | 40% | 0 | 40-50 | 5 | 5 | 1 |
| | 50% | 0-10 | 60 | 5 | 5 | 1 |
| 2 | 10% | 10-25 | 10-25 | 2 | 2 | 5 |
| | 20% | — | 40-50 | 2 | 3 | 5 |
| | 30% | — | 30-40 | 3 | 3 | 5 |
| | 40% | — | 45-60 | 3 | 5 | 5 |
| | 50% | 0-30 | 30-50 | 5 | 5 | 5 |
| 3 | 10% | 0 | 0 | 1 | 1 | 1 |
| | 20% | 0 | 35-45 | 1 | 1 | 2 |
| | 30% | 0 | 40-50 | 2 | 1 | 3 |
| | 40% | 0 | 50-70 | 3 | 2 | 3 |
| | 50% | 0-20 | 45-70 | 3 | 2 | 3 |
| 4 | 10% | 0 | 10-30 | 1 | 1 | 1 |
| | 20% | 0-10 | 50-60 | 1 | 1 | 2 |
| | 30% | 0 | 70-90 | 1 | 2 | 2 |
| | 40% | 0 | 80-90 | 2 | 2 | 3 |
| | 50% | 0-10 | 100-110 | 3 | 2 | 3 |

As can be seen, the use of ferromagnetic pigments in an anhydrous formula without latex polymer (Base 1) produces no curl at low use levels of iron oxides, and shows only moderate gains even at 50% iron oxide concentrations. With very low levels of latex polymer (Base 2), there is very little difference in curl as compared to the formulas without latex (Base 1). Further, the water resistance evaluations plummet to highly undesirable levels. It is only when the intermediate levels of latex polymer are used (Base 3) that improvements in curl in the 20-50% iron oxide concentration ranges are seen. Surprisingly, the Base 3 formulas also see a significant improvement over Base 2 with regards to water resistance, and significant improvements over Base 1 with regards to sebum and flake resistance. Finally, at high levels of latex polymer use (Base 4), there are startlingly high degrees of curl at 20-50% iron oxide concentration ranges, as well as increased water, sebum, and flake resistances over Base 2 and 3. Thus, Bases 3 and 4 produced enhanced curl with acceptable or better water, sebum, and flake resistances, in the covered ranges.

Formulas that lack the high melting point wax do not show the same surprising results as far as increased curl and good water, sebum, and flake resistance.

The unexpected performance of the composition when the latex polymers were present in amounts of at least 6% by weight, the ferromagnetic pigments (and specifically, iron oxides) were present in amounts of at least 20% by weight, and the compositions contained a high melting point wax, and especially when the ratio of the weight of all latex polymers to the weight of all waxes is between 0.76 and 1.0, can be seen in a comparison of the evaluations of the 50% iron oxide concentrations for each base formula. When the latex polymer is first added at low use levels, the results are incredibly poor—Base 2 performs substantially the same or worse in every evaluation metric. Base 4, however, provides very good results in the resistance metrics, and almost doubles the amount of curl experienced by the eyelashes.

Those skilled in the art will recognize or be able to ascertain using no more than routine experimentation many equivalents to the specific embodiments of the invention described herein. Such equivalents are intended to be encompassed by the following claims.

What is claimed is:

1. A method for improving the curl of an eyelash, comprising:
(a) applying onto an eyelash an aqueous cosmetic composition comprising:
one or more iron oxides in a total amount of at least 20% by weight;
one or more latex polymers present in an amount such that the total solids content of all latex polymers is at least 6% by weight; and
one or more waxes having a melting point Tₘ≥80° C.; and
(b) within a first period of time after applying the aqueous cosmetic composition, moving a magnetic element to a position above the eyelash in the direction of desired curl.

2. The method according to claim 1, further comprising repeatedly moving the magnetic element in a direction substantially parallel to the orientation of the eyelash while the magnetic element is above the eyelash in the direction of curl.

3. The method according to claim 1, further comprising repeatedly moving the magnetic element in a direction substantially perpendicular to the orientation of the eyelash while the magnetic element is above the eyelash in the direction of curl.

4. The method according to claim 1, wherein the first period of time is less than 30 seconds.

5. The method according to claim 1, wherein the ratio of the weight of all latex polymers to the weight of all waxes is between 0.76 and 1.0.

6. The method according to claim 1, wherein the one or more latex polymers comprise a styrene/acrylates/ammonium methacrylate copolymer.

7. The method according to claim 1, wherein the one or more latex polymers are present in an amount between 8% and 16% by weight.

8. The method according to claim 7, wherein the one or more iron oxides are present in an amount between 20% and 50% by weight.

9. The method according to claim 1, wherein the one or more latex polymers are present in an amount of at least 10% by weight.

10. The method according to claim 9, wherein the one or more latex polymers are present in an amount of at least 12% by weight.

11. The method according to claim 1, wherein the one or more waxes with a melting temperature above 80° C. are present in a total amount of at least 1% by weight.

12. The method according to claim 1, wherein at least one of the one or more iron oxides is uncoated.

13. The method according to claim 1, wherein the aqueous cosmetic composition further comprises at least one cellulosic polymer, natural fiber, synthetic fiber, or a combination thereof.

14. The method according to claim 1, wherein the aqueous cosmetic composition further comprises at least one additional colorant.

15. The method according to claim 1, wherein the aqueous cosmetic composition further comprises at least one filler agent.

16. The method according to claim 1, wherein the aqueous cosmetic composition further comprises at least one surfactant.

17. The method according to claim 1, wherein the aqueous cosmetic composition further comprises at least one preservative.

18. The method according to claim 1, wherein the aqueous cosmetic composition further comprises at least one wax having a melting point Tₘ<80° C.

19. A kit for shaping eyelashes, comprising:
(a) an aqueous cosmetic composition comprising:
one or more iron oxides in a total amount of at least 20% by weight;
one or more latex polymers present in an amount of at least 6% by weight; and
one or more waxes having a melting point Tₘ≥80° C.; and
(b) a magnetic element adapted to allow a user to magnetically interact with the aqueous cosmetic composition when the aqueous cosmetic composition is applied to an eyelash; and
(c) optionally instructions for use.

20. An aqueous cosmetic composition for eyelashes, comprising:
(a) one or more iron oxides in a total amount of at least 20% by weight;
(b) one or more latex polymers present in an amount of at least 6% by weight; and
(c) one or more waxes having a melting point Tₘ≥80° C.
